(19) United States
(12) Patent Application Publication
(10) Pub. No.: US 2002/0146714 A1
(43) Pub. Date: Oct. 10, 2002

(54) Direct haplotyping using carbon nanotube probes

(76) Inventors: Charles M. Lieber, Lexington, MA (US); Adam T. Woolley, Orem, UT (US); Jong-In Hahm, Somerville, MA (US); David Housman, Newton, MA (US)

Correspondence Address: FOLEY HOAG LLP, PATENT GROUP, 155 SEAPORT BOULEVARD, BOSTON, MA, 02110, US

(21) Appl. No.: 09/951,133

(22) Filed: Sep. 12, 2001

Related U.S. Application Data
(60) Provisional application No. 60/231,608, filed on Sep. 11, 2000.

(51) Int. Cl. C12Q 1/68; C12P 19/34

(52) U.S. Cl. 435/6; 435/91.2

(57) ABSTRACT

A method is described for multiplexed detection of polymorphic sites and direct determination of haplotypes in DNA fragments, DNA, and genomic DNA, using single-walled carbon nanotube (SWNT) atomic force microscopy (AFM) probes. This technique has applications for haplotyping in population-based genetic disease studies and other genomic screening.

### RELATED APPLICATION INFORMATION

This application claims the benefit of priority to Provisional Patent Application No. 60/231,608, filed Sep. 11, 2000, which application is hereby incorporated by reference in its entirety.

### GOVERNMENT SUPPORT

This invention was made with support from the Air Force Office of Scientific Research; the government, therefore, has certain rights in the invention.

### BACKGROUND OF THE INVENTION

The Human Genome Project is now providing massive amounts of genetic information that should revolutionize both the understanding and diagnosis of inherited diseases. In particular, the cataloging of single nucleotide polymorphisms (SNPs) in gene coding and regulatory regions should lead to a greater comprehension of the genetic contribution to risk for common diseases such as cancer and heart disease. To achieve maximum power, the haplotype of a subject-the specific alleles associated with each chromosome homologue—is a critical element in SNP mapping. However, the current methods for determining haplotypes have significant limitations that have prevented their use in large-scale genetic screening. For example, parental genotyping can be used to infer haplotypes in a family study, although in many cases it is impractical or impossible to obtain parental DNA. Furthermore, molecular techniques for determining haplotypes, such as allele specific or single molecule PCR amplification, are hampered by the need to optimize stringent reaction conditions and the potential for significant error rates.

Current sequencing methods may lead to expanded SNPs databases, and it has been proposed that such databases could be used in whole-genome association and linkage studies to determine the many interacting genetic effects that determine susceptibility to diseases such as cancer. Recent studies suggest, however, that the exploitation of SNPs for determining disease risk and pharmacogenomics will not be so easy. There are several reasons for emerging difficulties, including that most of the SNPs identified are likely unrelated to gene function (coding and/or regulatory regions). Without complete knowledge of all SNPs, which is a daunting task when one considers the costs of sequencing control and patient genomes from different population groups, direct association may not yield robust information about disease risk. There are additional considerations as well. For example, an individual heterozygous at n-sites (SNPs), could have 2n possible haplotypes, and each of these haplotypes may exhibit subtle variations in phenotype.

These latter observations suggest that the haplotype of a subject-the specific alleles associated with each chromosome homologue-is a critical element in SNP mapping. However, the current methods for determining haplotypes have significant limitations that have prevented their use in large-scale genetic screening. For example, parental genotyping can be used to infer haplotypes in a family study, although in many cases it is impractical or impossible to obtain parental DNA. Furthermore, molecular techniques for determining haplotypes, such as allele specific or single molecule PCR amplification are hampered by the need to optimize stringent reaction conditions and the potential for significant error rates. These latter methods are also difficult to apply to repeat sequences, which can be an important component of regulatory regions of genes.

Current methods of SNP mapping based on polymerase chain reactions (PCR) and fluorophore-labeled detection involve multiple biomedical reaction steps which result in a complicated and costly detection process, i.e., a few dollars per base, and these restrictions hinder their use for identifying polymorphisms in long DNA. Furthermore, these methods are not conducive for direct genomic DNA (gDNA) analysis. Moreover, this conventional method of SNP detection typically provides information about genotype, but genotyping alone does not contribute enough specifics to unambiguously determine the functional abnormalities of a gene. Haplotype determination is a decisive measurement in SNP mapping since haplotyping of a subject—differentiating the specific alleles associated with each chromosome homologue—provides the most incisive and complete information for understanding genetic contributions. Conventional methods for determining haplotypes require acquiring complete parental genetic information in a pedigree which is extremely difficult to obtain in practice. This requirement intensifies difficulties experienced by the traditional haplotyping methods through introducing additional limitations to the pre-existing complications due to the need for allele-specific or single molecule PCR amplifications. Haplotyping is conventionally achieved by deduction since circumstances are frequently left with insufficient genetic resources from a pedigree and, thereby, this inferring method is prone to errors. Straightforward, PCR-free haplotyping is highly warranted in large-scale genetic screening for population studies but not practical at present.

Hence, there appears to be a clear need for new technology that can determine directly sample haplotype with a high throughput.

### SUMMARY OF THE INVENTION

The present invention is directed to haplotyping by direct visualization of polymorphic sites on individual biosequences. According to one aspect of the invention, a method is disclosed for detecting single nucleotide polymorphisms of gene samples using an atomic force microscope (AFM). In one embodiment, the method of detecting single nucleotide polymorphisms of gene samples comprises the steps of a) scanning a nanotube tip connected to the AFM across the gene sample, b) acquiring an image of the gene sample and c) analyzing the acquired image to detect a single nucleotide polymorphism of the gene sample.

In an embodiment, the gene sample comprises DNA. In a further embodiment, the gene sample comprises a DNA fragment. . In an embodiment, the gene sample comprises an amplified DNA fragment using polymerase chain reaction. In another embodiment, the gene sample is genomic DNA (gDNA). The method can be used to read directly multiple polymorphic sites in DNA fragments containing from about 100 to at least 10,000 bases, at least 15,000 bases, or even at least 20,000 bases

In an embodiment, the biosequence or gene sample may comprise a probe to detect a single base mismatch e.g. single nucleotides polymorphorisms, or more than one single base mismatches in a gene. In certain embodiments, the probe corresponds to a molecule, biosequence, or a genetic or peptide process which creates a feature which is detectable by atomic force microscopy. In certain further embodiments, the probe is a peptide nucleic acid (PNA). In certain other embodiments, the probe is an oligonucleotide.

In an embodiment, the probe comprises a label or detectable moiety. In a further embodiment, an oligonucleotide probe is labeled with a molecule which can be detected by atomic force microscopy. In a further embodiment, the oligonucleotide probe is labeled with streptavidin. In an embodiment, the gene is gDNA. In a further embodiment the gDNA is labeled with a peptide nucleic acid probe. In an even further embodiment, the gDNA is labeled with a peptide nucleic acid probe:with SEQ ID 1: 5′-CTTTATGCCACAGAGCTA-3′. In yet another embodiment, the gene sample contains more than one label or probe.

In another embodiment, the gene has more than one single nucleotide polymorphism. In a further embodiment, the single nucleotide polymorphisms further comprise probes. In an even further embodiment, the probes comprise different peptide nucleic acids.

In an embodiment, the throughput of this method can be extended, for example, by employing arrays of multiple SWNT tips.

In another embodiment, the nanotip comprises a single walled nanotube (SWNT). In a further embodiment, the nanotube tip comprises multiple SWNTs. In another further embodiment, the nanotube tip comprises an individual SWNT.

Another aspect of the invention is directed to an atomic force microscope comprising a scanning probe having at least one nanotube tip, a scanner coupled to at least one of the scanning probe and a support for a gene sample, the scanner operating to scan the scanning probe relative to the gene sample on the support, a detection system for measuring the deflection of the nanotube during the scanning of the scanning probe relative to the gene sample, and an imaging system coupled to the detection system, the imaging system generating an image of a characteristic feature of at least a portion of the gene sample based on the deflection measurements from the detection system, the characteristic feature corresponding to at least one of a height and a spatial extent of the gene sample. The characteristic feature corresponds to a height and/or a spatial extent of said at least one sample portion, wherein said sample is a gene sample. In a further embodiment, the gene sample further comprises a probe.

The tips of the AFM may employ high-resolution single-walled carbon nanotube (SWNT) probes, which can be reproducibly prepared with tip radii of less than 3 nm and about 2 base resolution, about 5 base resolution, or about 10 base resolution to enable high resolution, multiplex detection of different labels.

In one embodiment, specific hybridization of labeled oligonucleotide probes may be used to target sequences in DNA fragments, using PCR, followed by direct reading of the presence and spatial locations of the labels by AFM. In this embodiment, the oligonucleotide probes are designed such that under appropriate hybridization conditions, binding does not occur in the presence of a single-base mismatch at polymorphic sites; i.e., labels are detected only at sequences fully complementary to the oligonucleotides. The oligonucleotides can be labeled with differing size molecules or compounds, which can be consistently distinguished from one another on the basis of size.

In another embodiment, peptide nucleic acids (PNA) are used as probes. In a further embodiment, the peptide nucleic acids interact with double stranded DNA (dsDNA) for haplotyping which can be detected by visualization of the labeled DNA using AFM with a SWNT tip.

In another embodiment, the gene is gDNA. In a further embodiment, the gDNA is comprises a PNA probe. In yet another embodiment, gDNA comprises a PNA and is separately labeled. In a further embodiment, gDNA comprises more than one PNA which have differing labels. In an even further embodiment, gDNA is labeled with both biotin- and fluorescein-terminated PNAs. In an even further embodiment, gDNA comprises a biotinylated PNA which is labeled with streptavidin-magnetic beads. In a further embodiment, the SWNT tip comprises an individual SWNT.

In another embodiment, the apparatus further comprises a micron scale channel which elongates biosequences such as DNA along a well-defined direction. In a further embodiment, the micron scale channel comprises polydimethylsiloxane (PDMS).

### BRIEF DESCRIPTION OF THE DRAWINGS

FIG. 1A shows schematic diagram of an atomic force microscope and image display.

FIG. 1B shows schematic diagram of an curved silicon tip and a cross sectional view.

FIG. 2A depicts a fragment of a stick model of a single-walled carbon nanotubes.

FIG. 2B depicts a transmission electron micrograph of a cross-sectional structure of multi-walled carbon nanotube.

FIG. 2C depicts a transmission electron micrograph of a cross-sectional structure of single-walled carbon nanotube.

FIG. 3A shows schematically a Fe-catalyzed CVD growth process of nanotubes using ethylene.

FIG. 3B shows a CVD apparatus for controlled growth of nanotubes.

FIG. 4 shows a schematic illustration of the surface growth process used to prepare SWNT tips.

FIGS. 5A and B depict scanning electron microscope images of the CVD nanotube tip before (A) and after (B) electrical etching.

FIGS. 5C shows a TEM image of the end of a shortened nanotube;

FIG. 6A illustrates schematically one method of detection of labeled DNA sites with nanotube tips: labeled oligonucleotide probes are specifically annealed to their complementary target sequences (a and b) but not to sequences with a single base mismatch (A and B) in the ssDNA template.

FIG. 6B illustrates one method of depositing labeled DNA molecules on freshly cleaved mica and imaging by AFM using SWNT probes.

FIG. 7A shows SWNT tip AFM image and height profile along DNA, obtained with streptavidin-labeled GGGCGCG in M13 mp18 digested with BglII; the arrow points to the streptavidin tag; the image height scale is 3 nm and the white bar corresponds to 100 nm.

FIG. 7B shows a histogram of number of streptavidin tags as a function of distance from the BglII restriction site, obtained from height plots along M13mp18 labeled at GGGCGCG.

FIG. 7C shows a histogram of number of streptavidin tags as a function of distance from the BglII restriction site, obtained from height plots along M13mp18 labeled at GGGCGCG, where the sample was digested with BamHI and distances were measured from this site.

FIG. 7D shows possible positions of the target sequence, based on the calculated distance from the restriction sites; the labeled sequence occurs where two arrowheads meet (one from each digest); solid arcs indicate the correct paths, while incorrect paths are shown as dashed arcs with an “x” through them.

FIG. 8A illustrates schematically direct haplotyping of UGT1A7 using SWNTs and shows haplotypes, alleles, genotypes, and locations of probes in samples analyzed.

FIG. 8B depicts representative SWNT tip AFM height image of the *3 allele (streptavidin end-labeled, ˜140 nm DNA) detected in a sample that was heterozygous at both loci.

FIG. 8C depicts SWNT probe image of the *1 allele (IRD800 end-labeled, ˜210 nm DNA) detected in the same sample as 8B; the height scale is 3 nm and the white bar corresponds to 50 nm in both images.

FIG. 8D depicts a histogram showing number of streptavidin (blue) and IRD800 (red) end-labeled fragments vs. DNA length for a sample known to have either the (*1/*3) or (*2/*4) haplotype.

FIG. 8E depicts a histogram same as in 8D, but from a different individual; both samples were determined to have the (*1/*3) haplotype.

FIG. 9A depicts a schematic of an approach to SNP and haplotype characterization by directly imaging peptide nucleic acid (PNA)-labeled and elongated DNA fragments where bold vertical lines represent each chromosome homolog and the letters specify the two polymorphic sites.

FIG. 9B depicts schematic representations of PNA labels targeting 129K131K and 208R sites in the * 1/*3 and *2/*4 haplotypes of UGT1A7 gene.

FIG. 10A depicts AFM images of a PNA labeled UGT1A7 gene.

FIG. 10B depicts AFM images of doubly-labeled UGT1A7 gene; the PNA labels are visible in the left image and the right image shows further conjugation with streptavidin with the maximum height difference between PNAs and dsDNA is about 0.4 nm. Inserts in the AFM images are the height profiles taken along the lines defined by the white arrows. A sample shown in the top histogram illustrates two clusters of PNA labels which are separated by the average distance of 81.3 nm (˜239 base pairs).

FIG. 10C depicts a histogram representing the PNA label distribution on the gene taken from another individual, showing the presence of both labels at their expected locations, and displaying the characteristics of the *1/*3 haplotype.

FIG. 11A depicts AFM images of m13mp18 reacted with PNAs (guided with white arrows in the left image) and additional streptavidin labels (right image) by targeting 5′-ACGCGCC-3′ and 5′-TCTCAGCC-3′ sites; the two labels are identified at the average distance of 0.28 μm and 1.42 μm from the restriction site which corresponds to the estimated positions of the 5′-ACGCGCC-3′ (0.26 μm) and 5′-TCTCAGCC-3′ (1.44 μm) sites from the restriction site.

FIG. 11B depicts AFM images of m13mp 18 reacted with streptavidin to the biotinylated ends of each PNA probe, 5′-TCTCAGCC-3′ (A) and 5′-ACGCGCC-3′ (B), showing sequence-specificity of PNAs.; each label is identified at their expected locations of 0.27 μm and 1.41 μm from the restriction site as shown in the combined histogram plot.

FIG. 12A depicts a schematic representation of a PDMS microfluidic channel

FIG. 12B depicts an AFM image of elongated m13mp18 on a silicon substrate using the controlled flow within a PDMS channel of 100 μm in width.

FIG. 12C depicts an AFM image of aligned lambda DNA on a silicon substrate using the controlled flow within a PDMS channel of 100 μm in width.

FIG. 12D depicts elongated, singly-labeled m13mp 18 by conjugating streptavidin to the biotinylated PNA labels.

FIG. 13 depicts automated haplotype determination using multiple SWNT tip arrays.

FIG. 14 schematically depicts haplotyping with genomic DNA.

FIG. 15 schematically depicts a cystic fibrosis transmembrane conductance regulator (CFTR) mapping using a PNA reaction of gDNA, and labeled with streptavidin coated paramagnetic particles, and captured by a magnet.

FIG. 16 depicts the PNA labels for SNP/haplotype mapping of the cystic fibrosis gene.

FIG. 17 shows AFM images of a SNP site labeled with streptavidin magnetic beads-biotin-PNA (5′-CTTTATGCCACAGAAGCTA-3′) and PNA (5′-GTCCGCTAG-3′)

FIG. 18 shows an AFM image of the Delta F508 site in a CFTR gene separated from gDNA

FIG. 19 shows examples of multiplexing in CFTR.

### DETAILED DESCRIPTION

The present invention is directed to haplotyping by direct visualization of polymorphic sites on individual genes. According to one aspect of the invention, a method is disclosed for detecting single nucleotide polymorphisms of gene samples using an atomic force microscope (AFM). In one embodiment, the method of detecting single nucleotide polymorphisms of gene samples comprises the steps of a) providing the AFM with at least on nanotube tip, b) moving said nanotube tip across the gene sample, and c) recording an image obtained with said AFM. In an embodiment, the nanotube tips are comprised of single-walled nanotubes (SWNTs). In an embodiment, metal-catalyzed chemical vapor deposition (CVD) is used to achieve reproducibility in the growth characteristics of the nanotube tips.

In an embodiment, the gene comprises a probe. A probe may include a nucleotide sequence such as an oligonucleotide which targets a specific sequence in a gene, for example in DNA or in a DNA fragment. A probe may also include a peptide nucleic acid. In one embodiment, two or more probes are used. For example, two probes may be designed to hybridize to a target sequence thereby generating a detectable signal whereby the probing nucleobase sequence of each probe comprises the complement to about half of the complete target sequence.

In a further embodiment, the probe comprises a label or detectable moiety which can be attached to an probe to render the probe detectable by AFM. Such labels may include derivatized avidin ,streptavidin, large monodisperse labeling proteins, a dextran conjugate, a branched nucleic acid detection system, a chromophore, a fluorophore, for example, 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye, a spin label, a radioisotope, an enzyme, a hapten, for example, 5(6)-carboxyfluorescein, 2,4-dinitrophenyl, digoxigenin, and biotin, an acridinium ester, chemiluminescent compounds, and magnetic labels. Other labels include heteroduplexes. In another embodiment, the probe may be hybridized to a nucleic acid target to cause a detectable change in at least one physical property of at least one attached label in a manner which can be used to detect, or identify, the presence of a single nucleotide polymorphism.

In a further embodiment, spacer or linker moieties may be used for example, to minimize the adverse effects that bulky labeling reagents might have on hybridization properties of probes. Linkers may induce flexibility and randomness into the probe or otherwise link two or more nucleobase sequences of a probe or component polymer. Examples of spacer/linker moieties which may be used for the nucleobase polymers of this invention consist of one or more aminoalkyl carboxylic acids (e.g. aminocaproic acid) the side chain of an amino add (e.g. the side chain of lysine or ornithine) natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane). Spacer/linker moieties may also incidentally or intentionally be constructed to improve the water solubility of the probe

The probes need not be labeled with a detectable moiety. When using probes, it is possible to detect the probe/target sequence complex formed, for example, by hybridization of the probing nucleobase sequence of the probe to the target sequence.

One or more of the probes may be immobilized to a surface for the detection of SNPs. In one embodiment, two or more probes may be immobilized each at a specified position. Because the location and composition of each immobilized probe is known, arrays are generally useful for the simultaneous detection, or identification, of SNPs of two or more gene sequences. Arrays of probes may be regenerated by stripping the hybridized nucleic acid after each assay, thereby providing a means to repetitively analyze numerous samples using the same array.

In another aspect, an apparatus for detecting single nucleotide polymorphisms of a gene, comprising an atomic force microscope with a scanning probe having at least one nanotube tip, scanning means for scanning said nanotube tip across a sample, detection means for detecting with said nanotube tip a characteristic feature of a least a portion of the sample, said characteristic feature corresponding to height and/or a spatial extent of least one sample portion, wherein said sample is a gene sample. In one embodiment, the characteristic feature is a probe or label on the gene. In a further embodiment, the sample comprises more than one characteristic feature.

### Definitions

The term “biosequence” refers to a gene, genetic or protein sequence, peptide nucleic acid, or DNA or DNA fragment.

The term “genomic DNA” or “gDNA” generally refers to the entire length of DNA, including non coding regions.

As used herein, the terms “label” and “detectable moiety” shall be interchangeable and shall refer to moietes which can be attached to a nucleotide, oligonucleotide or PNA probe to render the probe detectable by AFM.

As used herein, the term “nucleobase” shall include those naturally occurring and those non-naturally occurring heterocyclic moieties commonly known to those who utilize nucleic acid technology or utilize peptide nucleic acid technology to thereby generate polymers which can sequence specifically bind to nucleic acids.

The term “nucleobase sequence” shall include any segment of a polymer which comprises nucleobase containing subunits. Non-limiting examples of suitable polymers or polymers segments include oligonucleotides, oligoribonucleotides, peptide nucleic acids, nucleic acid analogs, nucleic acid mimics or chimeras.

A peptide nucleic acid (PNA) is a non-naturally occurring polyamide which can hybridize to nucleic acid (DNA and RNA) with sequence specificity (U.S. Pat. No. 5,539,082 and Egholm et al., Nature 365: 566-568 (1993)). Being a non-naturally occurring molecule, unmodified PNA is not known to be a substrate for the enzymes which are known to degrade peptides or nucleic acids. Therefore, PNA should be stable in biological samples, as well as have a long shelf-life. The hybridization of a PNA with a nucleic acid is fairly independent of ionic strength and is favored at low ionic strength, conditions which strongly disfavor the hybridization of nucleic acid to nucleic acid (Egholm et al., Nature, at p. 567).

The terms “polynucleotide,” “oligonucleotide,” “nucleic acid” and “nucleic acid molecule”, and “DNA” are used herein to include nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. These terms refer only to the primary structure of the molecule. Thus, these terms include, for example, 3′-deoxy-2′, 5′-DNA, oligodeoxyribonucleotide N3′P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

The term “probe” refers to a biosequence which targets sequences in for example genes, DNA and DNA fragments. The probing nucleotide or nucleobase sequence of a probe may be the specific sequence recognition portion of the construct.

As used herein, the term “target sequence” is a nucleic acid nucleobase sequence to which a least a portion of the probing nucleobase sequence is designed to hybridize.

### Atomic Force Microscope

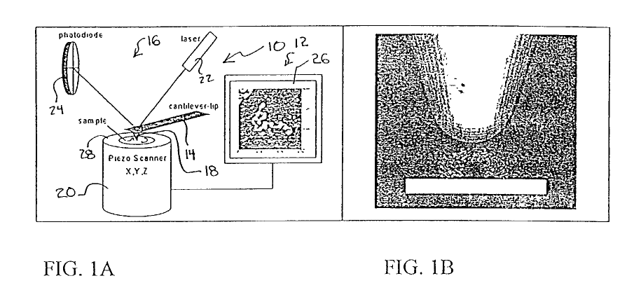

A schematic diagram of an atomic force microscope (AFM ) 10 is depicted in FIG. 1A. The AFM 10 can include a scanning probe 14, a detection system 16, and an imaging system 12. The scanning probe 14 is in the form of the cantilever and includes a nanotube 18 that is attached to an end of the scanning probe 14. A scanner 20 operates to move a sample, such as a gene sample, relative to the nanotube 18. The scanner 20 can be coupled to a support holding the sample or, alternatively, the scanner 20 can be coupled to the scanning probe 14 to move the scanning probe 14. The detection system 16 can measure the deflection of the nanotube 18 during the scanning of the scanning probe 14 relative to the sample. The detection system 16 can be an optical deflection system that includes a light source 22, such as a laser, and an optical sensor 24, such as a photodiode. The imaging system 12 is coupled to the detection system 16 to receive deflection measurements from the detection system 16. The imaging system 12 includes a processor (not shown) for generating an image of a characteristic feature of at least a portion of the sample based on the deflection measurements from the detection system 16. The characteristic feature can be, for example, at least one of a height and a spatial extent of the sample. The imaging system 12 can include a display device 26 for displaying the generated image.

During operation, the sample probe (cantilever) 14 obeys Hooke's Law for small displacements and the interaction force between the nanotube tip 18 and the sample may be found. The scanner 20 controlling the movement of the tip 18 or sample can be an extremely precise positioning device made from piezo-electric ceramics, such as a tube scanner. A support 28 for the sample can be the top surface of the scanner 20 or can be a separate support structure coupled to the scanner 20. The scanner 20 is preferably capable of sub-angstrom resolution in x-y- and z-directions. The z-axis is conventionally perpendicular to the sample.

The ATM can operate in at least one of three modes: a contact mode, a tapping mode and a non-contact mode.

In the contact mode, the tip 18 and sample remain in close contact as the scanning proceeds. The term “contact” refers to the repulsive regime of the inter-molecular force curve. One of the drawbacks of remaining in contact with the sample is that there exist large lateral forces on the sample as the tip 18 is “dragged” over the sample.

In the tapping mode, the sample probe 14 (cantilever) is oscillated at its resonant frequency (often hundreds of kilohertz) and positioned above the surface of the sample so that it only taps the surface for a very small fraction of its oscillation period. The reduced time over which this tapping occurs dramatically reduces the lateral forces on the tip 18 and the sample as the tip 18 scans over the surface. When imaging poorly immobilized or soft samples, tapping mode may be a far better choice than contact mode for imaging.

Other methods of obtaining image contrast are also possible with tapping mode. In a constant force mode, the feedback loop adjusts so that the amplitude of the sample probe (cantilever) 14 oscillation remains nearly constant.

FIG. 1B shows a scale model of a 5 nm radius of curvature silicon tip and a cross sectional view. The diameter of duplex DNA would correspond to the roughly circular black space surrounded by the GroES macromolecule and substrate (light rectangle).

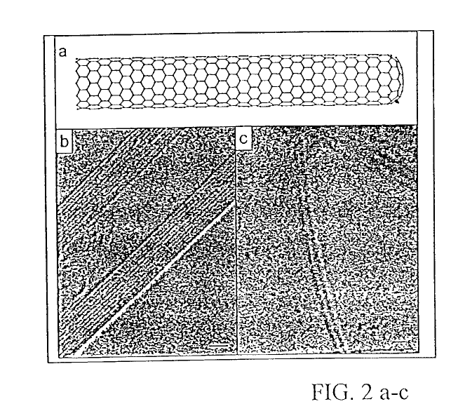

To exploit the intrinsic sensitivity and simplicity of AFM for DNA sequence detection, molecular scale tips are required that can be produced reproducibly. Carbon nanotubes consist of a honeycomb sp2 hybridized carbon network (termed a graphene sheet) that is rolled up into a seamless cylinder (FIGS. 2a-c), which can be microns in length. There are two basic structural classes of carbon nanotubes: single-walled nanotubes (SWNTs) and multi-walled nanotubes (MWNTs). SWNTs consist of a single seamless cylinder (FIG. 2a) with radii ranging from 0.35 to 2.5 nm, while MWNTs consist of multiple concentric graphene cylinders with radii ranging from 3-50 nm. Transmission electron microscopy (TEM) images of an individual MWNT (FIG. 2b) and SWNT (FIG. 2c) show clearly the tubular structure of the nanotubes, and in the case of the SWNT (FIG. 2c) highlight the extremely small diameter and high aspect ratio, which makes this material uniquely suited as a high-resolution probe tip.

Nanotubes have exceptional mechanical properties. The characteristics relevant to the use of nanotubes as AFM tips are the stiffness or Young's modulus and the ability to buckle elastically under large loads. Experimental measurements of Young's moduli made by recording thermal vibration amplitudes in a TEM and by direct bending with AFM revealed Young's moduli of 1.8 TPa and 1.3 TPa, respectively, showing that nanotubes are stiffer than any other known material. The extremely high Young's modulus of nanotubes is desirable for the creation of high aspect ratio, sub-nanometer radius tips with high resolution—if the modulus was significantly smaller, then the amplitude of thermal vibrations would degrade the resolution of tips. Carbon nanotubes buckle elastically under large loads, unlike conventional materials that either facture or plastically deform. MWNTs and SWNTs have been observed in the buckled state by TEM, and appear to buckle in a manner strikingly similar to macroscopic tubes made of elastic materials. The first direct experimental evidence that carbon nanotube buckling is elastic came from an early use of nanotubes as AFM tips. The buckling force of the nanotube tip could be measured from the deflection of the AFM cantilever as the nanotube tip-surface separation was reduced. The nanotube would then return to its original configuration when the tip was removed from the surface. A more direct measurement of elastic nanotube buckling was achieved by AFM measurements of force versus displacement of nanotubes pinned at one end on a surface. Both types of experiments demonstrate that nanotubes can be bent close to 90-degrees many times without observable damage, and thus are highly robust probes for AFM imaging.

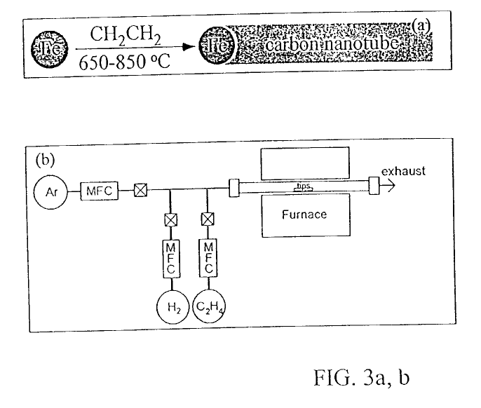

Carbon nanotube tips may be prepared by a direct synthesis of tips on standard atomic force microscopy cantilever/tip assemblies using metal-catalyzed CVD. In this process (FIGS. 3a, b), a metal particle catalytically decomposes a hydrocarbon feedstock and nucleates the growth of a carbon nanotube. This feature of the CVD process used for preparing nanotube probes provides conditions for the reproducible growth of SWNT tips having a consistent size and resolution and hence predictable characteristics.

Tips synthesized using the electrochemically deposited iron catalyst may consist reproducibly of individual 3-5 nm radii MWNTs oriented optimally for high-resolution imaging. Significantly, these studies demonstrated that a well-defined synthetic approach could be used to prepare directly nanotube probes, thus opening the possibility of precise control over nanotube size and thereby tip resolution. Recently, SWNT tips having much smaller radii of only 1-3 nm were reproducibly grown using well-defined iron oxide nanocluster catalysts. These latter tips begin to approach the theoretical minimum size expected for individual SWNTs. These tips may be prepared and attached as described in co-pending application U.S. 60/233595 incorporated herein by reference in its entirety.

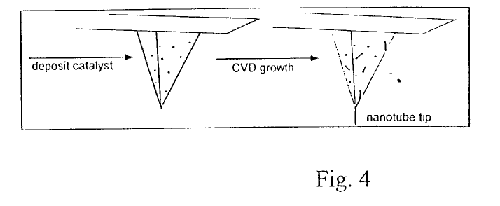

One process for producing CVD SWNT tips involves direct growth of SWNTs tips from the pyramid of a silicon cantilever-tip assembly shown in FIG. 4. In this ‘surface growth’ approach, the trade-off between the energy gain of the nanotube-surface interaction and the energy required to bend the nanotubes facilitates the reproducible growth of SWNTs from the silicon pyramid apex in the ideal orientation for high resolution imaging. This result can be understood by considering the fate of a nanotube during growth. When a growing nanotube reaches an edge of the pyramid, it can either bend to align with the edge or protrude from the surface. The pathway followed by the nanotube is determined by a trade-off in the energetic terms introduced above: if the energy required to bend the tube and follow the edge is less than the attractive nanotube-surface energy, then the nanotube will follow the pyramid edge to the apex; that is, nanotubes are steered towards the tip apex by the pyramid edges. At the apex, the nanotube must protrude along the tip axis since the energetic cost of bending is too high.

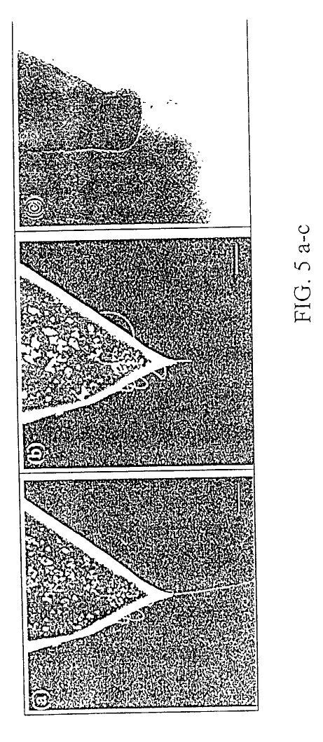

This steering of nanotubes to the pyramid apex to form ideal probe tips has been demonstrated experimentally (FIG. 5). For example, SEM investigations of nanotube tips produced by the surface growth method show that a high yield of tips contain nanotubes only at the apex, with very few protruding elsewhere from the pyramid (FIG. 5). These tips, which have been produced under conditions expected to yield SWNTs, are sufficiently small so that SEM cannot provide an accurate measure of their diameters. TEM analysis was able to demonstrate that the tips consist of individual SWNTs and small SWNT bundles. In the case of the small SWNT bundles, the TEM images show that the bundles are formed by nanotubes coming together from different edges of the pyramid to join at the apex, thus confirming the surface growth model described above (FIG. 5c).

The surface-growth approach for preparing individual SWNT tips appears to advantageously lower the catalyst density on the surface such that only one nanotube reaches the apex. This approach may be readily extended to prepare multiprobe arrays. The reproducible growth of individual SWNT tips, which represent a unique and heretofore unavailable technology for molecular level detection and imaging, advantageously allows the detection and visualization of polymorphic sites on individual DNA molecules with single nucleotide sensitivity, in particular the high-resolution detection of specific DNA sequences in kilobase fragments and haplotyping of a putative cancer related gene with two polymorphic sites, as will be described in more detail below.

Well-defined iron nanoclusters may be used as a catalyst for CVD growth of nanotubes. The motivation for the use of preformed iron nanoclusters is two-fold. Catalysts with a well-defined diameter can control the diameter of nanotubes and nanowires and can lead to more reproducible initiation of active nanotube growth. The catalyst clusters can be made by solution phase synthesis, as a means of achieving greater control over growth. Another significant factor in defining the reproducibility of SWNT tip growth is dispersion of the catalyst on a micro-fabricated silicon pyramid, which serves as the support during surface growth. In general, the catalyst can be dispersed uniformly over the entire pyramid. This approach, however, can cause large fluctuations in the distribution of catalyst near the apex at the low catalyst concentrations used to achieve individual SWNTs, which can reduce reproducibility. To mitigate fluctuation and also enable a more efficient deposition of catalyst, a dual-purpose, semi-automated manipulation stage can be used for catalyst deposition on Si pyramids and DNA sample deposition on substrates. In this approach, the catalyst can be deposited in a defined outer region of the silicon pyramid through controlled transfer of a catalyst ‘inked’ elastomeric polydimethylsiloxane (PDMS) stamp. This can be achieved by depositing a thin, uniform layer of iron nanocluster catalyst onto a flat PDMS block by spin coating, and then bringing the pyramid into contact with the PDMS using the manipulation stage. Two characteristic features of this approach are that (1) it can enable confinement of the catalyst to the outer 100 nm or less of the pyramid and (2) it can enable controlled batch processing of tip arrays, which is advantageous for large scale SWNT synthesis and for the creation of multiprobe arrays.

The individual SWNT tips require relatively tight constraints on tip length to avoid loss of resolution due to thermal vibrations. It has been found that the amplitude of the tip vibration as a function nanotube length for several discrete diameters for a radius of an individual SWNT tip of 0.5 nm, the length will need to be on the order of 10-20 nm to avoid loss of resolution. In other words, the nanotube length has to be precisely controlled to take advantage of the potential resolution enhancements of individual SWNT tips. One method for controlling the length is an electrical etching method—by determining the voltage dependence—which may allow control of the tip shortening process on a nanometer scale. In one embodiment, pulsed electrical etching can be used to remove portions of the nanotube, wherein the amount removed by pulsed electrical etching depends directly on pulse height, with the length being reproducibly reduced in steps of about 2 nm or larger. The current shortening process hence provides sufficient control to optimize the length of individual SWNT tips for imaging.

In an embodiment, the SWNT tip length is controlled by direct growth; that is, defining the tip length by the growth time. This approach requires control of initial nucleation, growth rate and positioning of the catalyst, which all can impact the reproducibility of the growth of individual SWNT. In one approach, nanotubes can be grown on planar substrates which enables rapid analysis of the resulting nanotube for a predetermined set of conditions.

It will be understood by those skilled in the art that the sample throughput can be increased at the image acquisition stage using parallel versus serial detection, for example, by employing multiprobe tip arrays for imaging. In addition, various modifications of the AFM can be implemented, such as high resonant frequency (>100 kHz) cantilevers in combination with fast system electronics to increase the potential image acquisition rate.

An aspect of the present invention is the preparation and deposition of DNA samples or biosequences for simultaneous characterization and haplotyping of two or more distinct single nucleotide variations and detection of SNPs in large DNA samples. Since the AFM tip performs a controlled movement across the DNA molecule, elongation and alignment of DNA is desirable that are compatible with SWNT tip imaging. In one embodiment, reproducible sequence detection in large samples is attained by elongating the DNA to eliminate strand crossings, which could produce false positive signals.

Within one embodiment, the fluid flow at the edge of evaporating drops is exploited to elongate DNA. This approach produces a radial alignment of the molecules on surfaces. This approach may also be used for SWNT AFM imaging using DNA samples in the 10-100 kb length range. In addition, the manipulation device, which was described above, may also be used for producing small periodic arrays of elongated samples, which can automate sample preparation.

The evaporating droplet approach to DNA elongation cannot readily predict alignment of DNA relative to the AFM scan axes. Preferential alignment of multiple DNA samples with respect to one of the scan axes, however, can be advantageous for increasing throughput; that is, image data could acquired much more quickly for a high aspect ratio rectangle (n×m pixels with n>>m) window versus a square (n×n) window. Another known approach for achieving both DNA elongation and preferential alignment involves the use of microfluidic structures. This approach utilizes a controlled laminar flow within a micron scale PDMS channel to elongate (flow) and align (unidirectional flow direction) the DNA in a defined manner.

### Method A

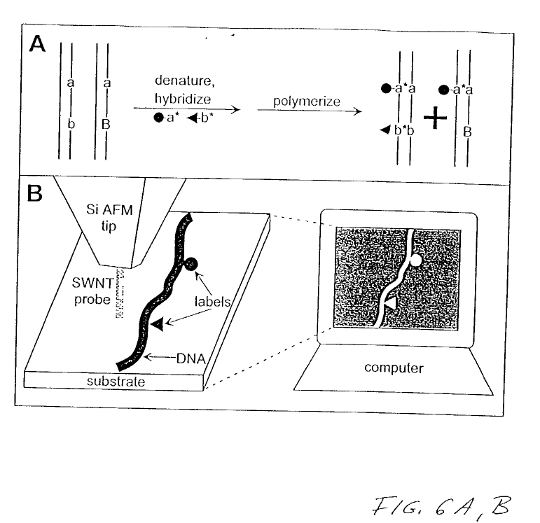

In one embodiment, specific hybridization of labeled oligonucleotide probes may be used to target sequences in DNA fragments, followed by direct reading of the presence and spatial locations of the labels by AFM.(FIG. 6) The oligonucleotide probes are designed such that under appropriate hybridization conditions, binding does not occur in the presence of a single-base mismatch at polymorphic sites; i.e., labels are detected only at sequences fully complementary to the oligonucleotides. SWNT tips having tip radii of less than 3 nm were used, providing about 10 base resolution, for high resolution, multiplex detection of different labels. The exemplary oligonucleotides were labeled with either streptavidin or the fluorophore IRD800, which can be consistently distinguished from one another on the basis of size.

FIG. 6a shows labeled oligonucleotide probes ( -a* and -b*) that are specifically annealed to their complementary target sequences (a and b), but not to sequences with a single-base mismatch (A and B) in the single-stranded DNA template. DNA polymerase and dNTPs are then used to synthesize complementary strands, generating double-stranded DNA fragments specifically labeled at a and b with and , respectively. FIG. 6b shows labeled DNA molecules deposited on freshly cleaved mica and imaged by AFM using SWNT probes. The presence and locations of the sequence specific tags (and) can be readily observed in the AFM image. It is apparent that due to the size of the tip, only the SWNT tip (left), and not the conventional Si tip (right), can be expected to provide sufficient spatial resolution.

Direct haplotyping using SWNT AFM probes appears to represent a significant advance over conventional approaches and could facilitate the use of SNPs for association and linkage studies of inherited diseases and genetic risk. These methodologies may be used to detect multiple SNPs in about 10 kilobase samples with a resolution of approximately 10 bases, and moreover, could be extendable to about 100 kilobase samples with similar resolution. The large DNA sizes that can be directly haplotyped are unique to the SWNT AFM technique and may be useful independent of the sample throughput. In addition, the simplicity and distinctiveness of the AFM images of alternative haplotypes indicate that automated analysis may also be feasible. The current throughput for an instrument imaging with a single SWNT tip may be greater than 200 samples/day with a redundancy of about 10 independent images per sample. In addition, this approach could be extended from single sample analysis to a very high throughput parallel technique by exploiting multiple tip arrays, which have been fabricated in sizes as large as about 32×32 for ultrahigh density hard-disk storage. The implementation of these technical improvements would allow haplotyping of over about 200,000 samples per day with a single instrument using our technique. Recent synthesis of carbon nanotubes with about 0.25 nm radii, which are smaller than the spacing between DNA bases, indicates that further improvements in nanotube probes, nanotube end labeling and/or DNA labeling methods could enable direct reading of the DNA sequence of fragments that are tens of kilobases in size.

### Method B

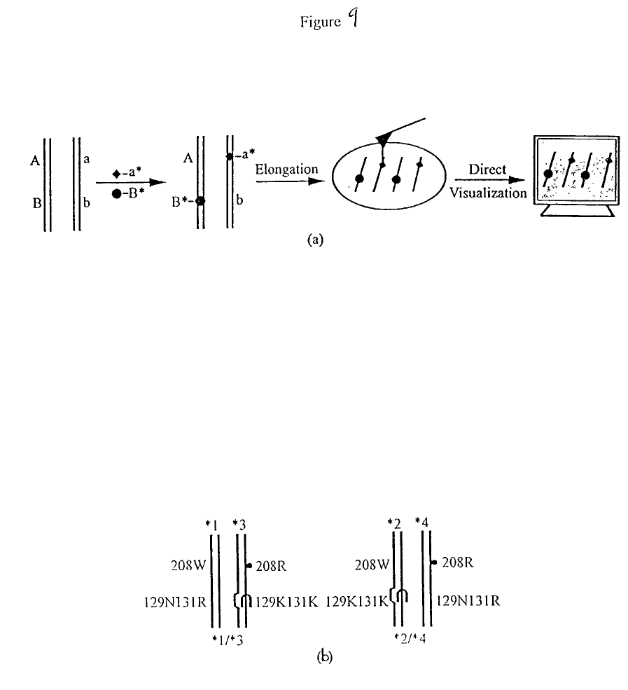

In another embodiment, the gene sample comprises peptide nucleic acids with double strand DNA. This embodiment is illustrated in FIG. 9a which describes a PCR-free PNA labeling scheme, the subsequent DNA elongation when necessary, and the label detection using an individual SWNT scanning probe. PNAs have shown great potential for use as probes to study single base mismatches in DNA as PNAs bind to dsDNA with high affinity, stability, and single base specificity. PNA is a DNA mimic in which the negatively-charged sugar backbone of DNA is replaced with an uncharged pseudo-peptide backbone.

PNA/DNA hybrids have higher stability than DNA/DNA duplexes due to the lack of the electrostatic repulsion between the two strands. The probing nucleobase sequence of a PNA probe is the specific sequence recognition portion of the construct. Therefore, the probing nucleobase sequence is a sequence of PNA subunits designed to hybridize to a target sequence to thereby be used to detect the presence, absence or number of organisms of interest in a sample. Consequently, with due consideration of the requirements of a PNA probe for the assay format chosen and the organism sought to be detected, the length of the probing nucleobase sequence of the PNA probe will generally be chosen such that a stable complex is formed with the target sequence under suitable hybridization conditions or suitable in-situ hybridization conditions.

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target combination is often found by the well known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

In one embodiment, the direct interaction of peptide nucleic acid (PNA) labels with double stranded DNA (dsDNA) in haplotyping which can be readily detected by simple visualization of labeled DNA using atomic force microscopy (AFM) with an individual SWNT tip. FIG. 9B clearly demonstrates the super sharp geometry of an individual SWNT tip which is fabricated on a commercial tip apex using the ‘pick-up’ method. Individual SWNT tips typically show diameters of less than the DNA width, thus minimizing tip broadening effects.

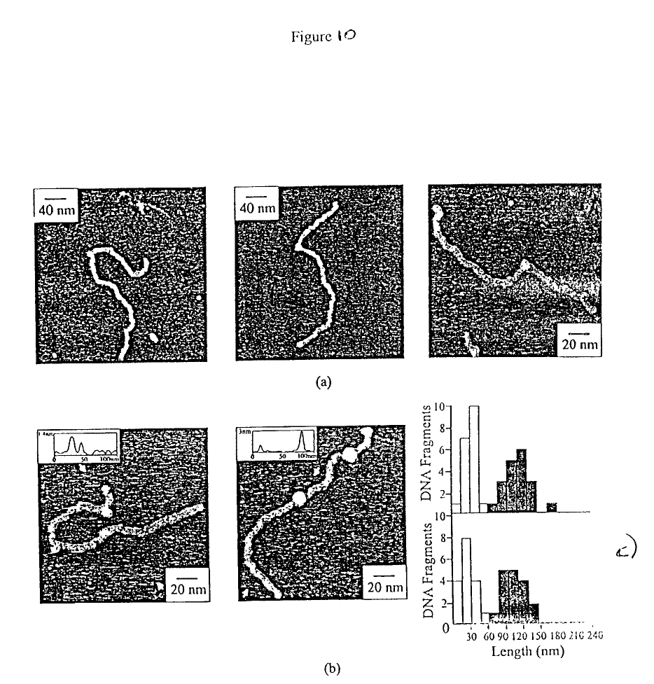

Detection of these PNA labels was accomplished by the use of individual SWNT scanning probes. SWNT probes provide routine high resolution imaging with the average tip diameter and aspect ratio of 2 nm and 10:1, respectively. Using these SWNT tips the DNA resolution may be full width at half maximum (FWHM) of ˜4 nm in width under typical ambient imaging conditions. PNA labels exhibit maximum height difference of only about 0.4 nm when compared to dsDNA whereas streptavidin labels conjugated to the biotinylated end of the PNA probes typically showed a change in height greater than 1.5 nm (FIG. 10B). Therefore, the successful use of PNAs in haplotyping, when accompanied by the utilization of individual SWNT probes, represents a significant advance towards a high-throughput technique for genomic screening. This ability to readily detect PNA labels enables a PCR-free, single-step labeling reaction which avoids lengthy PCR procedures for label conjugation to denatured DNA and signal amplification with streptavidin. It also expands the size range of label choices by allowing detection of multiplex labels with various sizes where the lower detection limit is approximately few tenths of nanometers in height. In one embodiment, this PCR-free labeling scheme employs PNA probes down to sizes as small as a 7-mer and incorporating an individual SWNT scanning probe to readily detect the PNA labels without the need of further conjugation with larger labels.

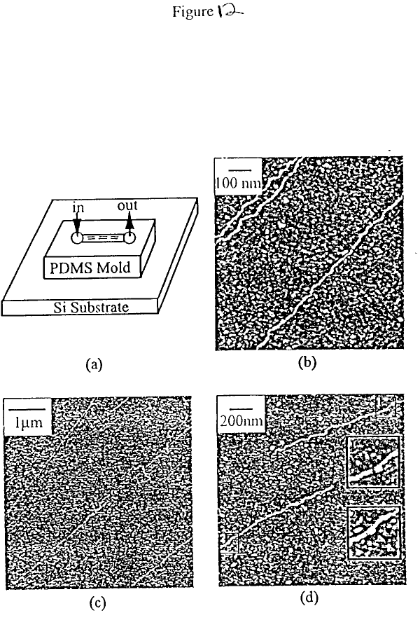

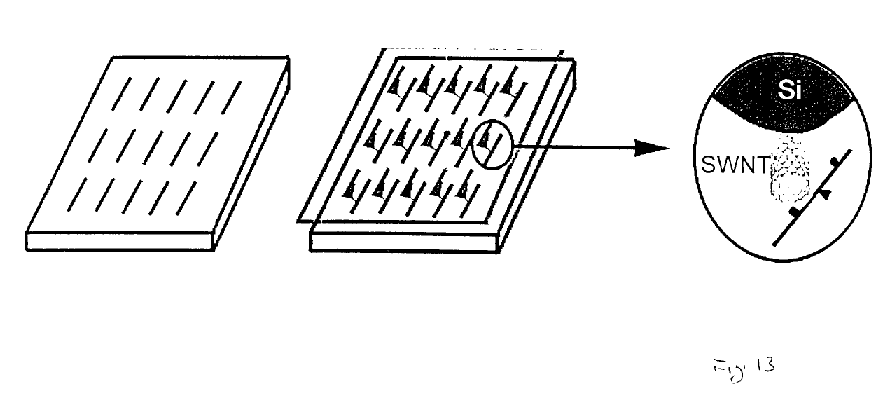

Difficulties in SNP mapping of long DNA with an AFM include bending, kinking, coiling or crossing of micron-long DNA strands when deposited on the mica surface, which can hamper accurate label detection and effective image analysis. The alignment of DNA strands of tens of microns in length is required to overcome this issue in our direct imaging approach. In one embodiment, long DNA fragments, up to ˜15 microns in length, are directionally aligned using controlled flow within micron-scale polydimethylsiloxane (PDMS) channel which can effectively elongate DNA along the flow direction, as illustrated in FIG. 12A. FIGS. 12B and 12C illustrate elongated m13mp18 and lambda DNA on a silicon/silicon oxide surface. This microfluidic alignment of DNA exhibit several advantages over the previous stretching techniques. The elongation of DNA is readily achieved without the use of centrifugal forces or electric fields and the alignment direction is well-defined. The directionality of aligned DNA can cooperatively enhance the efficiency of AFM detection. FIG. 4D shows labeled m13mp18 DNA fragments which were reacted with PNA and then further conjugated with streptavidin. Periodically aligned DNA deposition as well as fabrication of multi-tip arrays may promote the assembly of an integrated system with automated DNA sample deposition, image acquisition, and image analysis capabilities for effective and accurate haplotype determination.

### Method C

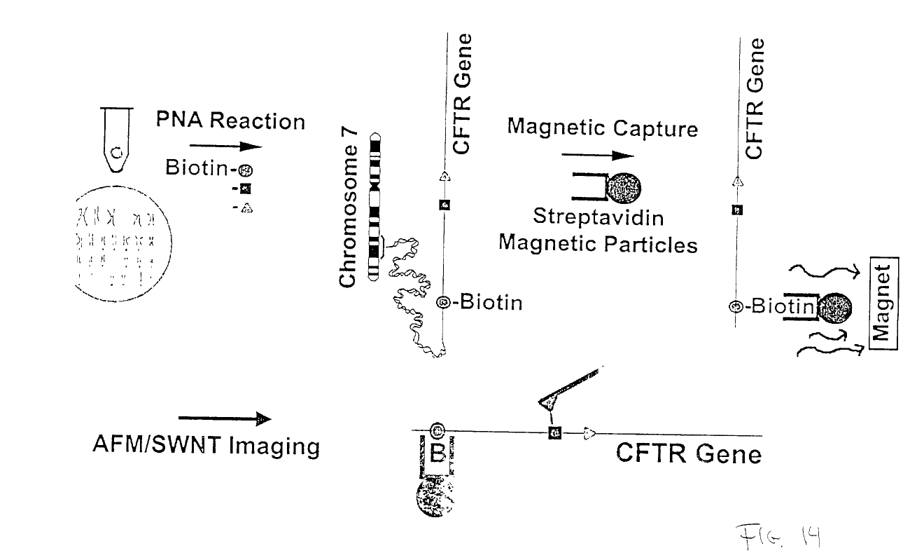

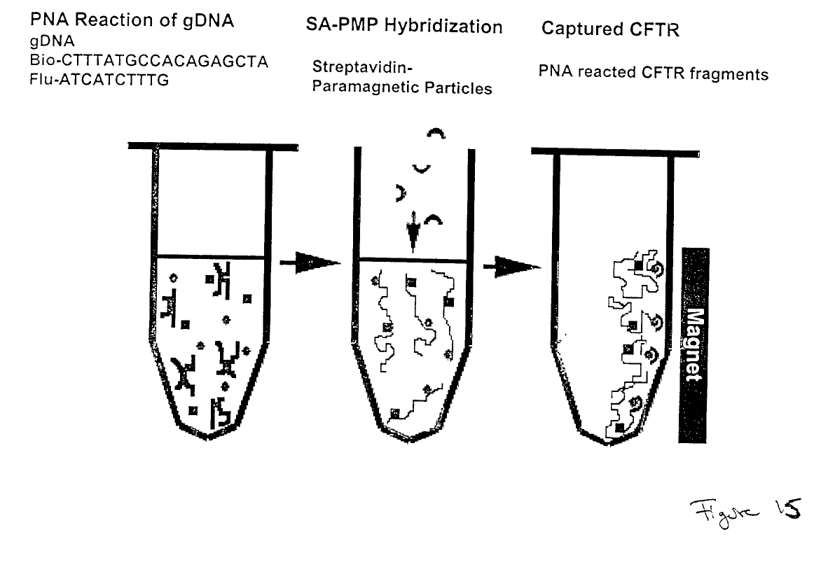

In yet another embodiment, SNP detection and haplotyping may be achieved on genomic DNA (gDNA) without amplifying the gene region. In one embodiment, single nucleotide polymorphism (SNP) haplotypes are determined in the cystic fibrosis transmembrane receptor gene, CFTR. The CFTR protein product is an ATP-binding cassette (ABC) transporter, functions as a chloride channel, and controls the regulation of other transport pathways. Homozygous mutations in this gene cause the respiratory disease cystic fibrosis In one embodiment, a biotinylated PNA tag is used to label specifically the genomic DNA pieces of CFTR. These maybe then optionally further conjugated with a moiety. In one embodiment, this moiety is streptavidin.. In another embodiment, further PNA labels may be employed to detect SNP sites. In a further embodiment, the moiety may be attached to super paramagnetic particles or labels, which may be used for separation purposes.(FIG.14 and 15)

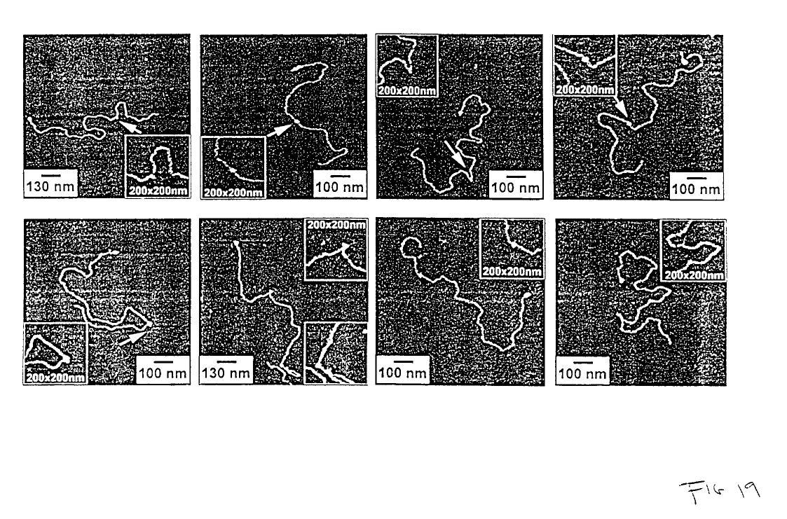

In another embodiment, the genomic DNA has more than one single nucleotide polymorphism. In a further embodiment, the single nucleotide polymorphisms further comprise probes. In an even further embodiment, the probes comprise different peptide nucleic acids. (FIG. 19)

Magnetic labels which may be used are beads or particles made either from nanometer-sized iron oxide crystallites, polymer impregnated with nanometer-sized iron oxide crystallites, or porous glass filled with iron oxide crystallites. These particles can be obtained with surface functional groups that may be used to immobilize molecules such as streptavidin, antibodies, or DNA. The particles are paramagnetic; that is, their magnetization is a function of the external magnetic field, and when the field is removed, the magnetization of the particles settles to zero. This “relaxation” does not happen instantly, but occurs over a period typically measured in microseconds or milliseconds, depending on the size of the iron oxide crystallites. Particles based on nanometer-sized iron oxide crystallites are sometimes termed “superparamagnetic”, since their magnetization in a given magnetic field tends to be much greater than normal paramagnetic materials.

Particles fabricated from ferromagnetic materials (such as NdFeB or nickel) or ferrimagnetic materials (such as micron-sized iron oxide or ferrite particles) can also be used as magnetic labels. Both types of materials can be magnetized to a substantially greater magnetic moment than superparamagnetic particles, but they also retain their magnetism in the absence of an external magnetic field.

After the reaction is completed, genomic DNA or DNA fragments of interest may be separated from other genomic DNA pieces using a magnet since the molecular tag in the reaction was hybridized with super paramagnetic particles. SNP and haplotype detection was then performed using SWNT scanning probes directly on the separated and also aligned genomic DNA fragments.

### EXEMPLEFICATION

The invention now being generally described, it will be more readily understood by reference to the following examples, which are included merely for purposes of illustration of certain aspects and embodiments of the present invention, and are not intended to limit the invention.

### Example 1

### UGT1A7 Alleles

The alleles of UGT1A7 with their Genbank accession numbers and genotypes at the polymorphic loci are UGT1A7*1, HSU39570 and HSU89507 (129N131R208W); UGT1A7*2, AF11019 (129K131K208W); UGT1A7*3, AF110192 (129K131K208R); UGT1A7*4, AF110193 (129N131R208R). The numbers and capital letters in the genotypes (i.e. 208R) correspond to the number and type of amino acid in the protein encoded by the UGT1A7 gene at the polymorphic site.

### Example 2

### PCR Amplification of UGT1A7 Samples

PCR primers (forward 5′-CTATCTGTACTTCTTCCACTTAC and reverse 5′-ACTTACATATCAACAAGAGCTGC) were designed to amplify a fragment which encompasses both polymorphic sites in the UGT1A7 first exon (from nt−76 to 1048 in the sequence corresponding to Genbank accession number HSU39570). PCR was performed on 20 ng of genomic DNA in 50 μl aliquots containing 20 pmol of each primer, 1× reaction buffer (50 mM KCl, 1.5 mM MgCl₂, and 10 mM Tris pH 8.5), 100 μM dNTPs, 4% DMSO, and 2 U Taq DNA polymerase (PE Applied Biosystems, Branchburg, N.J.), The amplification conditions were: denaturation at 94° C. for 5 min, 5 cycles each consisting of 60 s at 94° C., 45 s at 62° C. 72° C. followed by 30 cycles each consisting of 60 s at 94° C., 45 s at 56° C., 90 s at 72° C., followed by 7 min at 72° C. PCR products were purified using Qiagen quick columns (Qiagen, Santa Clarita, Calif.) to remove the primers and then dissolved in water.

### Example 3

### DNA Sequence Labeling in M13mp18.

### Method A

Biotin (Operon, Alameda, Calif.) or IRD800 (Li-Cor, Omaha, NE) labeled CGCGCCC (8 pmol) was annealed to 80 fmnol M13mp18 in 1× EcoPol buffer (New England Biolabs, Beverly, Mass.) with 100 μM dNTPs at 25° C. Klenow Fragment exo⁻ (New England Biolabs, 10 U) was added, allowed to sit at 25° C. for 5 min, and then warmed to 37° C. for 30 min. Restriction digestion was carried out on −30 fmol DNA with 5 U of BglII, BamHI or AlwNI in the recommended digestion buffer (New England Biolabs) at 37° C. for 60 min. Digesting two separate aliquots with a different restriction enzyme is necessary because DNA fragment ends are indistinguishable by AFM. After digestion biotin labels were conjugated with streptavidin (7.5 pmol) at room temperature for 10 min in restriction buffer. Samples were ethanol precipitated and resuspended in 10 mM Tris, 1 mM EDTA (TE), pH 8.0. Multiplex labeling at the sequence GCTGAGA was performed the same as described above, except the annealing was carried out at 15° C.

### Example 4

### Labeling UGT1A7 alleles

### Method A

PCR amplicons (˜100 fmol) were denatured at 95° C. for 10 min and then oligonucleotides (4 pmol) complementary to the 129N131R (IRD800-AATGACCGA) and 208R (biotin-AGTACGGAA) loci were annealed at 24° C. in 1× EcoPol Buffer with 50 μM dNTPs. Klenow Fragment exo^{—}(2.5 U) was added, and the mixture was maintained at 24° C. for 2 min, followed by heating to 37° C. for 30 min to extend the primed strands. Samples were purified with Concert PCR purification systems (Gibco BRL-Life Technologies, Grand Island, N.Y.) to remove excess primers, and the DNA was resuspended in TE, pH 7.0. Streptavidin (0.5 pmol) was conjugated to biotinylated DNA in TE with 0.1 M NaCl for 2 hr at room temperature.

### Example 5

### Sample Deposition and AFM Imaging

### Method A

DNA was diluted to ˜100 pg/μl in 10 mM MgCl₂ and deposited onto freshly cleaved mica for 5 min. Then the surface was rinsed several times with water and dried gently under a stream of nitrogen gas prior to AFM imaging. Images were recorded under ambient conditions in tapping-mode at 1.5-2 Hz with a tip resonance frequency of 60-70 kHz and amplitudes of 15-40 nm using a Digital Instruments (Santa Barbara, Calif.) Multimode Nanoscope IIIa. In contrast to previous contact-mode studies with micro-fabricated tips, the relative humidity was found to have a minimal influence on measured DNA heights in our experiments, and thus no efforts were made to control ambient humidity. The insensitivity to humidity is due to the very small cylindrical structure of the nanotube probes. SWNT ropes were mounted on Au-coated force modulation etched silicon probe cantilevers (Digital Instruments, k=1-5 N/m) using micromanipulators under the direct view of an optical microscope as described previously.

### Example 6

### Multiplexed Sequence Detection in M13mp18.

### Method A

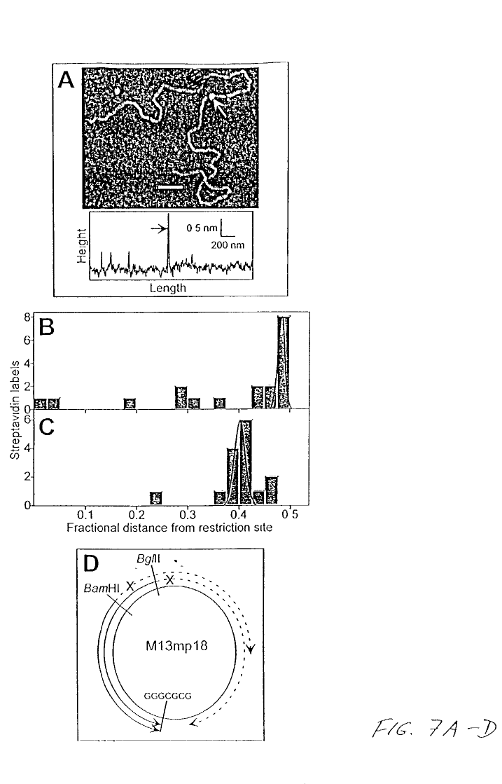

The method according to the invention of detecting haplotypes using SWNTs was employed to identify the spatial location of specific sequences with excellent discrimination from corresponding single-base mismatches in the M13mp18 plasmid using seven base oligonucleotide probes. The essence of this experiment is captured in the AFM image of a DNA molecule that was marked with a streptavidin-labeled GGGCGCG sequence, as shown in FIG. 7A. This image shows a DNA fragment with a 2200 nm contour length consistent with the 7249 bp of M13mp18, and a distinct streptavidin label 1080 nm from one end of the BglII digested DNA. The arrow points to the streptavidin tag. Places where DNA strands cross each other (left side of height profile) are easily differentiated from labels. The image height scale is 3 nm and the white bar corresponds to 100 nm.

FIGS. 7B shows a histogram summary of results obtained from at least 15 streptavidin-labeled M13mp18 DNA molecules showing a clear peak at 0.48 (3512 bp) from the BglII restriction site for samples cut with BglII. FIG. 7C shows a similar histogram summary for samples cut with BglI of results obtained from the at least 15 streptavidin-labeled M13mp18 DNA molecules showing a clear peak at 0.40 (2893 bp) from the BamHI restriction site. FIG. 7D shows a map of M13mp18 with the location of GGGCGCG calculated from the histograms of FIGS. 7B and 7C. Arrowheads indicate possible positions of the target sequence, based on the calculated distance from the restriction sites; the labeled sequence occurs where two arrowheads meet (one from each digest). Solid arcs indicate the correct paths, while incorrect paths are shown as dashed arcs with an “x” through them.

In contrast, histograms from control experiments with unlabeled oligonucleotides (data not shown) did not exhibit clusters of labels, indicating that the histogram peaks are due to specific detection of streptavidin. These results demonstrate two key points. First, based on the calculated distances of streptavidin labels from the two restriction site positions, the GGGCGCG site was determined to be at base 3390 (FIG. 7D). This same site was calculated to be at base 3402 from similar experiments with IRD800 labeling (data not shown); both results are in excellent agreement with the known location (base 3405). Second, there is no evidence for labeling the single-base mismatch sites located at 1115 and 3595, thus demonstrating the specificity of labeling and the potential for SNP detection.

Because the streptavidin and IRD800 molecules can be readily distinguished on the basis of their heights and shapes (e.g. average measured height of streptavidin labels was 1.7±0.5 nm vs. 0.7±0.3 nm for IRD800) using the SWNT tips, simultaneous detection of two or more distinct sites is feasible. To test this concept, M13mp18 labeled at GGGCGCG with IRD800 and at TCTCAGC with streptavidin was prepared, and these fragments were imaged using SWNT probes. Histograms similar to FIG. 7B, C were generated for streptavidin (>1 nm) and IRD800 (<1 nm) peaks detected in surface plots along imaged DNA fragments for BglII and AlwNI digests (data not shown). From these histograms, TCTCAGC was calculated to occur at bases 2024 and 4059, in good agreement with its known positions at 2013 and 4077, with GGGCGCG calculated to occur at base 3422, corresponding well with the expected value of 3405. These results demonstrate clearly the potential for multiplexed sequence detection in large DNA strands and open the possibility for profiling multiple polymorphic sites on DNA fragments in the 10 kilobase or larger size range.

### Example 7

### Direct Haplotype Determination in UGT1A7.

### Method A

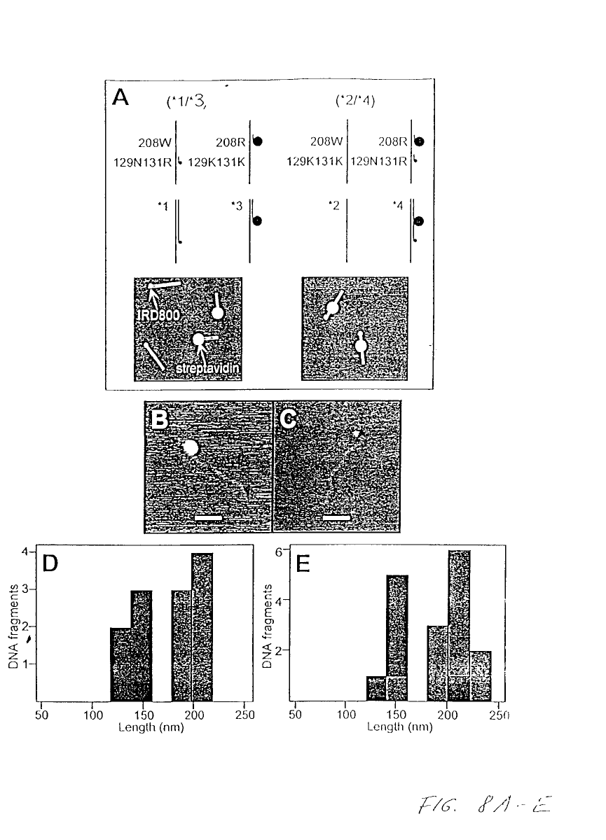

Referring now to FIGS. 8A-C, experiments were performed to identify specific haplotypes associated with genetic disorders. To illustrate this critical point, haplotypes were determined on a UDP-glucuronosyltransferase gene, UGT1A7, whose enzyme product is involved in inactivation of carcinogens such as benzo[a]pyrene metabolites, by using SWNT probes. This gene has two polymorphic sites (separated by 233 bp) that determine four alleles, each specifying different polypeptide chains. FIG. 8A shows schematically haplotypes, alleles, genotypes, and locations of probes in the analyzed samples. Individuals who are heterozygous at both sites have a single genotype. The (*1/*3) and (*2/*4) haplotypes, which have the same genotype, are specifically labeled at the 129N131R and 208R sites with IRD800 (small filled circle) and streptavidin (large filled circle), respectively. These haplotypes, (*1/*3) or (*2/*4), cannot be differentiated using conventional methods. This ambiguity is crucial in screening, since each allele exhibits substantially different enzymatic activity towards targeted carcinogens. To distinguish these haplotypes using SWNT tips, allele specific probes were hybridized to DNA samples (FIG. 8A). AFM images of a (*1/*3) sample should have an approximately equal number of fragments that are ˜210 nm (663 bp) long with IRD800 at one end, or ˜140 nm (430 bp) long with streptavidin at one end (the random coil structure of the single-stranded DNA tail should not contribute significantly to the length). In contrast, a (*2/*4) sample should contain ˜210 nm fragments with IRD800 at one end and streptavidin ˜70 nm (233 bp) distant. The probes were chosen such that the (*1/*3) haplotype would show singly labeled fragments while the (*2/*4) haplotype would exhibit DNA with two or no labels.

The subject samples could be unambiguously shown to exhibit the (*1/*3) haplotype by direct inspection of AFM images. FIG. 8B shows a representative SWNT tip AFM height image of the *3 allele (streptavidin end-labeled, ˜140 nm DNA) detected in a sample that was heterozygous at both loci. FIG. 8C shows a representative SWNT tip AFM height image of the *1 allele (IRD800 end-labeled, ˜210 nm DNA) detected in the same sample. DNA molecules were only end-labeled with the streptavidin or the IRD800 probes. These images are characteristic of the *3 and * 1 alleles, respectively. Control experiments on samples homozygous for the *2 allele showed no specific labeling (data not shown); because of the low occurrence frequency of the *4 allele, we did not test any samples known to carry this allele.

To further substantiate that the haplotype determined by image inspection was indeed the consequence of specific probe hybridization to the expected sites on the target DNA, detailed length measurements were carried out. The histograms depicted in FIGS. 8D, E plot the number of streptavidin and IRD800 end-labeled DNA fragments as a function of length, for two different samples, each showing a grouping for streptavidin around 140 nm and for IRD800 near 210 nm. The cluster of streptavidin tagged DNA at ˜140 nm is typical of the *3 allele, and the grouping of IRD800 labeled fragments at ˜210 nm indicates the *1 allele. These fragment distributions for both samples are in agreement with that expected for the (*1/*3) haplotype (FIG. 8A). Furthermore, no observed fragments matched the predicted profile of the *4 allele (FIG. 3A); hence, both of these samples were of the haplotype (*1/*3). FIG. 8E shows a similar histogram as FIG. 8D, except that the histogram of FIG. 8E is from a different individual; however, both samples were determined to have the (*1/*3) haplotype.

### Example 8

### PNA Labeling in UGT1A7 and M13m 18.

### Method B

PNA labels for UGT 1 A7, biotin-5′-TTCCGTACTA-3′ and biotin-5′-TTTTTT-Lys-Lys-TTTTTTGTCC-3′ were acquired from PE Applied Biosystems (Branchburg, N.J.). 0.1 μpmol of PNA was reacted to ˜1 nmol of UGT1A7 with 0.05M NaCl at 47° C. for 120 min. Further conjugation with streptavidin (10 pmol) was carried out, when necessary, in room temperature for 30 min. after ethanol precipitation of the reaction products. The same purification process was repeated to the final product and the sample was resuspended in 10 mM Tris, 1 mM EDTA (TE), pH 8.0. PNA labels with sequences of biotin-5′-ACGCGCC-3′ and biotin-5′-TCTCAGCC-3′ were purchased from PE Applied Biosystems to target m13mp18. Linearization of m13mp18 was carried out on 30 fmol DNA with 5 U of XbaI in the recommended digestion buffer (New England Biolabs) at 37° C. for 60 min. After digestion, the reaction mixture was purified using Qiagen quick columns and the purified DNA was redissolved in TE. PNA labels (0.1 μmol) were added with 0.05M NaCl and the mixture was incubated at 40° C. for 150 min. Samples were then ethanol precipitated and resuspended in TE, pH 8.0. PNA labels were conjugated with streptavidin (1 pmol) at room temperature for 30 min. Ethanol precipitation and resuspension in TE, pH 8.0 were repeated.

### Example 9

### DNA Alignment Using PDMS Microchannels

### Method B

A silicon master containing channel (100 μm in width and 3 mm in length) was produced using standard photolithography procedures.¹³ PDMS elastomer channels were produced from the master using Sylgard 184 cured at 70° C. for 180 min. PDMS elastomers seal conformally when they are brought into contact with poly-L-lysine (Ted Pella, Redding, Calif.) treated silicon oxide/silicon substrates. 2 μl of DNA in TE solution (˜1 pmol) was mixed with 5 μl of pure ethanol (Sigma Aldrich, Saint Louis, Mo.) immediately before the flow alignment. The typical flow rate and duration were 1 mm/s and 5 min. respectively. After the removal of the PDMS, the sample was rinsed with deionized water and dried with nitrogen gas.

### Example 10

### Sample Deposition and AFM Imaging

### Method B

DNA was deposited onto freshly cleaved mica for 5 min. with 10 mM MgCl₂. Then the surface was rinsed several times with deionized water and dried gently under a stream of nitrogen gas prior to AFM imaging. Aligned DNA samples prepared using silicon substrates were treated in the same way for the rinsing and drying. Images were recorded at 1.5-2 Hz with a tip resonance frequency of 60-70 kHz and an amplitude of 10-20 nm using a Digital Instruments (Santa Barbara, Calif.) Multimode Nanoscope IIIa. AFM tips with individual SWNT were prepared by the ‘pick-up’ method using FESP Si cantilevers (Digital Instruments, k=1-5 N/m).

### Example 11

### Haplotyping Using PNAs

### Method B

PNAs are used for haplotyping the UDP-glucuronosyltransferase gene, UGT1A7. The UGT1A7 gene ,the subject of extensive investigation due to its role in protection against benzo(a)pyrene- and aromatic amine-induced cancer, has two polymorphic sites which determine four alleles and specific combination of these alleles, haplotypes, are associated with potential risk factors which are caused by environmental carcinogens. Unambiguous determination of haplotypes of this gene, i.e., identification of alleles occurring for each copy of chromosomes, is therefore crucial to delineate the precise haplotypes associated with genetic disorders. AFM images in FIG. 10A clearly display PNA labels on the UGT1A7 gene which were consistently detected with SWNT tips.

Custom designed PNA probes were used to tag polymorphic sites of this gene, as shown in schematic representations in FIG. 9C. PNA probes were designed to delineate specifically the * 1/*3 haplotype from the *2/*4 haplotype which were found in individuals who are heterozygous at the two polymorphic sites having a single genotype, but have one of two haplotypes. Differentiation of these two haplotypes is particularly important since it cannot be readily determined by conventional methods. Two PNA probes, specifically aiming at 208R and 129K131K sites respectively, target DNA sequences that are fully complementary to the probe sequences under a proper reaction condition. Differentiation of the two haplotypes can be achieved by a direct visualization with AFM as well as by examining the presence and spatial locations of these PNA labels. For example, the * 1/*3 haplotype samples will show both labels on the same DNA fragment whereas the *2/*4 haplotype will exhibit a single label on each DNA fragment. Therefore, immediate distinction of the two haplotypes based on the simple visualization of the gene is possible with our detection method; DNA samples shown in the bottom AFM images of FIG. 10B have, for example, the *1/*3 haplotype.

Statistical analysis on the spatial locations of PNA labels on DNA strands were performed to substantiate accurate haplotype determination. A sample shown in the upper histogram in FIG. 9B displays two peaks of PNA labels which are separated by the average distance of 81.3 nm (239 base pairs). Their average positions were measured as 30.7 nm and 112.0 nm from one end of the gene with the average total length of 235.2 nm. The results show an excellent agreement with the actual locations of 129K131K (˜97 bp) and 208R (˜333 bp) sites on the gene where these two polymorphic sites are separated by ˜236 base pairs. Therefore it is determined that this individual's haplotype is *1/*3. The lower histogram shows the result of the PNA label distribution on the gene taken from another individual, showing the presence of both labels at their expected locations, and thus indicating the same haplotype for this individual. Allele-specific PNA interaction was repeatedly monitored in samples from other individuals as well (data not shown); 1) the UGT1A7 gene of 1123 bp in size was analyzed where only a single peak was resolved in the PNA label histogram. The position of the label, 206.7 nm from one end, confirms the existence of the 129K131K site but the absence of the 208R site, thus exhibiting the characteristic of a haplotype consisting of only *2 allele. 2) The PNA label distribution showed no clusters of labels when another DNA specimen was analyzed, indicating the *1/*1 haplotype.

### Example 12

### SNP Detection Using PNAs

### Method B

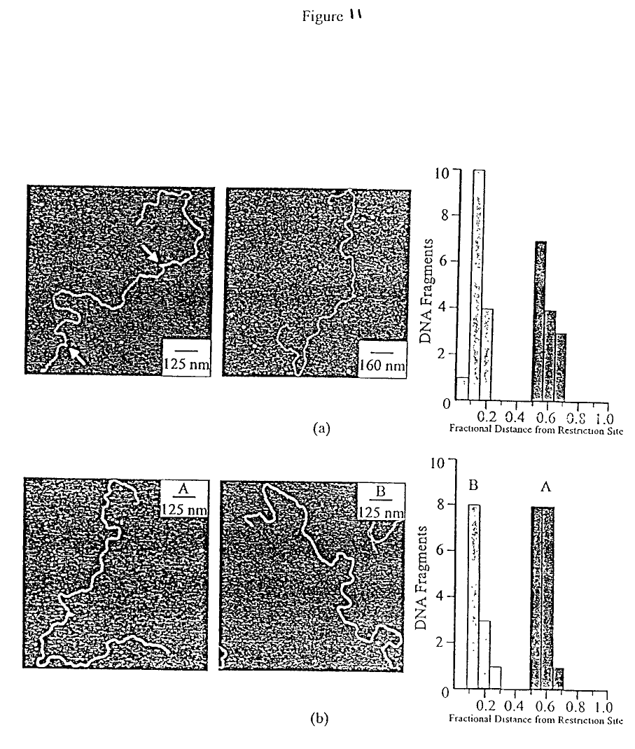

A model system, m13mp 18 and PNA probes with single base specificity were used to target specific sequences corresponding to single or multiple sites on linearized ml 3mp 18 whose average length was determined as 2.35 μm by AFM. Two PNA probes were employed to target 5′-ACGCGCC-3′ and 5′-TCTCAGCC-3′ sites respectively. Single base mismatch sites for these sequences are present at multiple positions on m13mp18. However, these PNA probes reacted only with the DNA sequences that were completely complementary to the probe sequence under appropriate reaction conditions and binding does not occur in the presence of a single-base mismatch at polymorphic sites, demonstrating the exceptional sequence-selectiveness of PNA probes in SNP mapping of DNA up to ˜7 kilobases in length. The histogram in FIG. 11 A shows that the two labels are located at the average distance of 0.28 μm and 1.42 μm from the restriction site which nicely corresponds to the estimated positions of the 5′-ACGCGCC-3′(0.26 μm) and 5′-TCTCAGCC-3′(1.44 μm) sites from the restriction site. Results shown in FIG. 11B further confirm the PNA efficiency in SNP mapping with its excellent discrimination from the existing single base mismatch sites.

### Example 13

### PCR Amplification of CFTR Alleles

### Method C

PCR primers (forward 5′-AGGGCATAGCTCTGTGGCATAAAG and reverse 5′-GAACTGTCTCTCAGGGGTATTTCTCC) were designed to amplify a 4265 bp fragment containing p art of introns 9 and 10, and the entire sequence for exon 10 (from nucleotide 10109 to 14374 in the sequence corresponding to GenBank accession number AC000111 ). PCR was performed on 50 ng of genomic DNA in 50 ul aliquots containing 20 pmol of each primer, 1×reaction buffer (40 mM Tri-HCL pH 9.3, 15mM KOAc, and 0.2% Triton X-100), 1.1 mM Mg(OAC)2, 100 uM dNTPs, and 1×Advantage Genomic DNA or 1×Advantage cDNA polymerase (Clontech, Palo Alto, Calif.). The amplification conditions were as follows: denaturation at 94° C. for 5 min, 40 cycles each of 15s at 94° C., 6 min at 68° C., followed by 10 min at 72° C. PCR products were gel purified on a 0.7% agarose gel followed by purification using Qiagen QIAquick columns (Qiagen, Santa Clarita, Calif.) to remove primers and then dissolved in 1mM Tris-HCl pH 8.5.

### Example 14

### PNA Labeling in Amplified CFTR.

### Method C

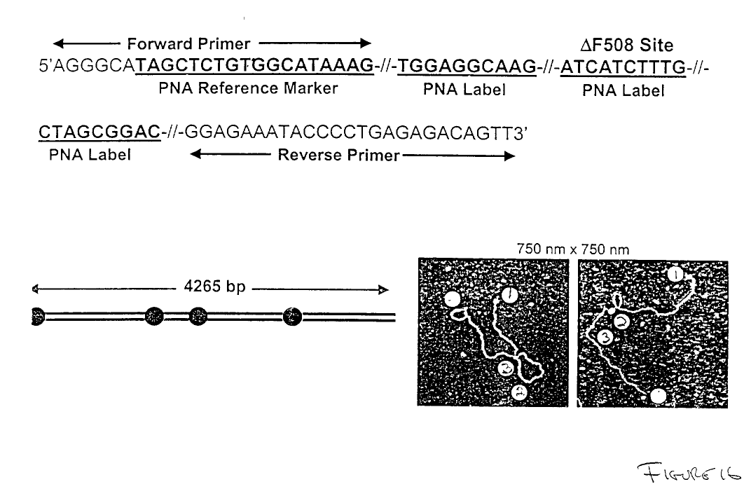

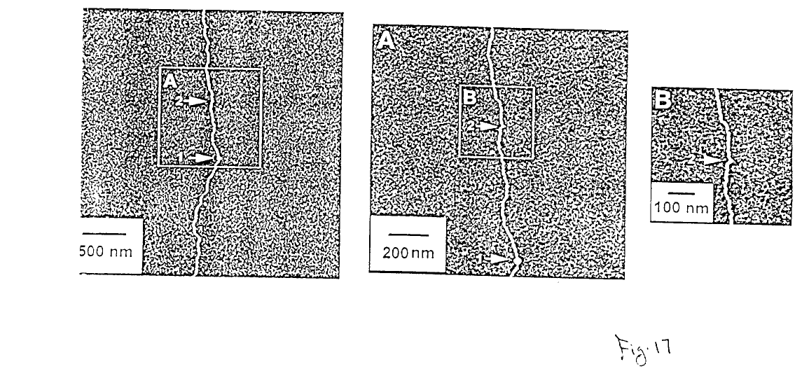

PNA labels for CFTR as shown below in Table 1 and FIG. 16 were acquired from PE Applied Biosystems (Branchburg, N.J.). 0.1 μmol of PNA was reacted to ˜10 nmol of CFTR with 0.1 M NaCl at 47° C. for 180 min. Further conjugation with streptavidin (10 pmol) was carried out, when necessary, in room temperature for 30 min. after ethanol precipitation of the reaction products. The same purification process was repeated to the final product and the sample was resuspended in 10 mM Tris, 1 mM EDTA (TE), pH 8.0.

| | |
|---|---|
| PNA Reference Marker | Biotin-5′-CTTTATGCCACAGAGCTA-3′ |
| PNA label for Site 1 | Biotin-5′-CTTGCCTCCA-3′ |
| | Fluorescein-5′-CTTGCCTCCA-3′ |
| PNA label for Site 2: | Biotin-5′-ATCATCTTTG-3′ |
| Delta F508 | Fluorescein-5′-ATCATCTTTG-3′ |
| PNA label for Site 3 | Biotin-5′-GTCCGCTAG-3′ |
| | Fluorescein-5′-GTCCGCTAG-3′ |

### Example 15

### PNA Labeling in Genomic DNA.

### Method C

Genomic DNA was obtained from Coriell Institute for Medical Research (Camdon, N.J.) and diluted in 10 mM Tris, 1 mM EDTA (TE), pH 8.0 to a concentration of ˜100 nmol. Two methods were used to randomly reduce sizes of gDNA fragments; 1) The solution was spun at 14000 rpm for 20 min prior to PNA labeling and 2) Digestion of gDNA was carried out with 2U of BglII in the recommended digestion buffer (New England Biolabs) at 37° C. for 60 min. After digestion, the reaction product was purified using Qiagen quick columns and the purified gDNA was redissolved in TE. 0.1 μmol of PNA was then reacted to gDNA with 0.1 M NaCl at 47° C. for 180 min. Streptavidin coated superparamagnetic particles were purchased from Promega Corporation (Milwakee, Wis.) and washed in pure ethanol three times using a magnetic stand (Promega Corporation, Milwakee, Wis.). PNA labeled gDNA was reacted with streptavidin coated superparamagnetic particles for 30 min at room temperature. After conjugation, the gDNA reaction mixture was kept overnight at 4° C. on a magnetic stand. Magnetically captured genomic DNA fragments were separated and aligned on a freshly cleaved mica substrate by spinning gDNA with MgCl₂ solution at 8000 rpm for 30 sec. The sample was then rinsed with deionized water and blow-dried under a gentle stream of N₂ gas. AFM images were recorded at 1.5-2 Hz with a tip resonance frequency of 60-70 kHz and an amplitude of 10-20 nm using a Digital Instruments (Santa Barbara, Calif.) Multimode Nanoscope IIIa. AFM tips with individual SWNT were prepared by the ‘pick-up’ method using FESP Si cantilevers (Digital Instruments, k=1-5 N/m).

### Example 16

### SNP Detection in Genomic DNA Pieces of CFTR

### Method C

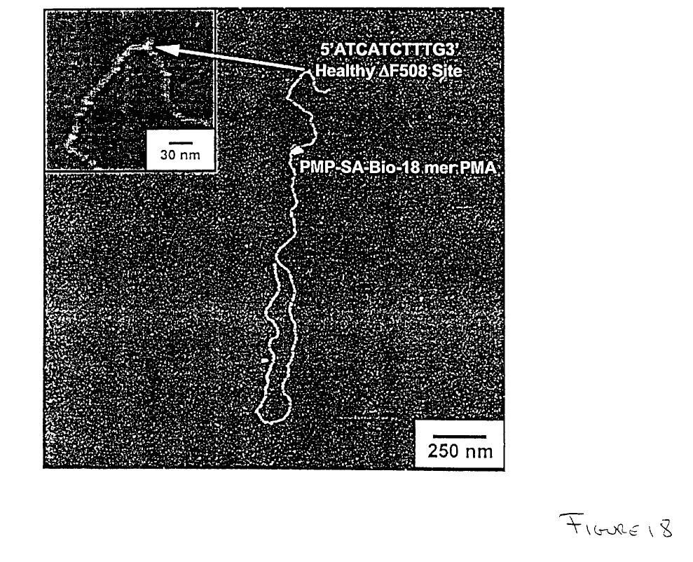

Genomic DNA is used without amplifying the gene region of CFTR. A biotinylated PNA tag is used to label specifically the genomic DNA pieces of CFTR which were then further conjugated with streptavidin. At the same time, other PNA labels were used to detect SNP sites, for example delta F508 site. (FIG. 18) The streptavidin in this case was attached to super paramagnetic particles and it reacts only with the biotinylated PNA tag which is specially designed such that it only binds with the CFTR gene. After the reaction was completed, genomic DNA fragments of CFTR was separated from other pieces using a magnet since the streptavidin in the reaction was hybridized with super paramagentic particles. SNP and haplotype detection was then performed using SWNT scanning probes directly on the separated and also aligned genomic DNA fragments.

### Example 17

### Multiplexing for Haplotyping Determination in CFTR

### Method C

Both biotin- and fluorescein-terminated PNA labels were used simultaneously in the reaction to target specific SNP sites of CFTR gene. The biotinylated PNA label was subsequently conjugated with streptavidin. This reaction resulted in multiple SNP sites which can be easily differentiated by AFM on the basis of different label sizes. (FIG. 19)

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of detecting a single nucleotide polymorphism of a gene sample using an atomic force microscope (AFM), comprising the steps of:
scanning a nanotube tip connected to the AFM across the gene sample,
acquiring an image of the gene sample, and
analyzing the acquired image to detect a single nucleotide polymorphism of the gene sample.

2. The method of claim 1, wherein the nanotube tip comprises a single walled nanotube.

3. The method of claim 2, wherein the nanotube tip comprises multiple single walled nanotubes.

4. The method of claim 2, wherein the nanotube tip comprises an individual single walled nanotube.

5. The method of claim 1, wherein the gene sample is a DNA fragment.

6. The method of claim 5, wherein the gene sample is a DNA fragment containing between 10 and 10,000 bases.

7. The method of claim 5, wherein the DNA fragment comprises an amplified DNA fragment using a polymerase chain reaction.

8. The method of claim 6, wherein the DNA fragment further comprises a probe.

9. The method of claim 6, wherein the DNA fragment further comprises an oligonucleotide probes designed to bind at sequences complementary to the oligonucleotides.

10. The method of claim 9, wherein said oligonucleotide probes further comprise a label which can be detected by atomic force microscopy.

11. The method of claim 10, wherein said label is streptavidin.

12. The method of claim 8, wherein the probe comprises a peptide nucleic acid.

13. The method of claim 12, wherein the peptide nucleic acid probe further comprises a label which can be detected by atomic force microscopy.

14. The method of claim 1, wherein the gene is genomic DNA.

15. The method of claim 14, wherein the genomic DNA further comprises a probe.

16. The method of claim 15, wherein the probe comprises a peptide nucleic acid.

17. The method of claim 16, wherein the genomic DNA further comprises a label which can be detected by atomic force microscopy.

18. The method of claim 14, wherein the genomic DNA comprises more than one probe.

19. The method of claim 18, wherein said probes are different.

20. The method of claim 18, wherein said probes are the same.

21. The method of claim 1, wherein said gene has more than one single nucleotide polymorphism.

22. The method of claim 21, wherein said single nucleotide polymorphisms further comprise probes.

23. The method of claim 22, wherein said probes comprise different peptide nucleic acids.

24. The method of claim 17, wherein said label comprises a paramagnetic label.

25. The method of claim 14, wherein the genomic DNA comprises a cystic fibrosis transmembrane receptor gene.

26. The method of claim 25, wherein the cystic fibrosis transmembrane receptor gene further comprises a peptide nucleic acid probe with the sequence given in SEQ ID 1.

27. The method of claim 1, wherein the nanotube tip has a radius of less than 3 nm.

28. The method of claim 1, wherein the AFM comprises arrays of single walled nanotube tips.

29. The method of claim 1, wherein the gene sample comprises an array of gene samples.

30. An atomic force microscope comprising:
a scanning probe having at least one nanotube tip,
a scanner coupled to at least one of the scanning probe and a support for a gene sample,
the scanner operating to scan the scanning probe relative to the gene sample on the support,
a detection system for measuring the deflection of the nanotube during the scanning of the scanning probe relative to the gene sample, and
an imaging system coupled to the detection system, the imaging system generating an image of a characteristic feature of at least a portion of the gene sample based on the deflection measurements from the detection system, the characteristic feature corresponding to at least one of a height and a spatial extent of the gene sample.

31. The apparatus of claim 30, wherein the nanotube tip is comprised of a single walled nanotube.

32. The apparatus of claim 31, wherein the nanotube tip is comprised of multiple single walled nanotubes.

33. The apparatus of claim 31, wherein the nanotube tip is comprised of an individual walled nanotube.

34. The apparatus of claim 30, wherein the gene sample further comprise a probe.

35. The apparatus of claim 34, wherein the gene sample comprise oligonucleotide probes.

36. The apparatus of claim 34, wherein the gene sample comprise peptide nucleic probes.

37. The apparatus of claim 34, wherein the gene sample are genomic DNA.

38. The apparatus of claim 30, wherein the nanotube tip has a radius of less than 3 nm.

39. The atomic force microscope of claim 30, further comprising micron scale channel formed in the support for the gene sample.

40. The apparatus of claim 29, wherein the AFM comprises arrays of single walled nanotube tips.
